(19) United States
(12) Patent Application Publication
(10) Pub. No.: US 2025/0221903 A1
(43) Pub. Date: Jul. 10, 2025

(54) HAIR CARE COMPOSITION

(71) Applicant: The Procter & Gamble Company, Cincinnati, OH (US)

(72) Inventors: Jennifer Mary MARSH, Deerfield Township, OH (US); Timothy James FELTS, Hamilton, OH (US)

(21) Appl. No.: 19/039,128

(22) Filed: Jan. 28, 2025

Related U.S. Application Data
(63) Continuation of application No. 17/209,292, filed on Mar. 23, 2021, now abandoned.
(60) Provisional application No. 63/000,016, filed on Mar. 26, 2020.

(51) Int. Cl. A61K 8/37; A61K 8/04; A61K 8/34; A61K 8/41; A61K 8/42; A61K 8/92; A61Q 5/12

(52) U.S. Cl. CPC A61K 8/375; A61K 8/042; A61K 8/342; A61K 8/416; A61K 8/42; A61K 8/922; A61Q 5/12; A61K 2800/5922

(57) ABSTRACT

A hair care composition comprising: a. from about 0.5% to about 5%, by weight of the composition, of a triglyceride oil having at least about 30% C18:1; b. a wax having a melting point of at most about 45° C.; c. one or more fatty alcohol; and d. a cationic surfactant system; wherein the composition comprises a gel matrix; and wherein the ratio of the triglyceride oil to the wax is from about 90:10 to about 50:50.

### FIELD OF THE INVENTION

The present invention relates to a hair care composition comprising oil and wax blends for natural hair conditioning.

### BACKGROUND OF THE INVENTION

A variety of approaches have been developed to improve the health of the hair. A common method of providing a hair health benefit is through the use of conditioning agents such as cationic surfactants and polymers, high melting point fatty compounds, low melting point oils, silicone compounds, and mixtures thereof. Most of these conditioning agents are known to provide various hair health benefits. However, some consumers would prefer to not use hair conditioners that have silicone compounds. This is either because of a desire to have more natural conditioning actives or because they do not like the feel that silicones deliver to their hair. Thus, there is a need for hair conditioners containing natural conditioning actives that can deliver consumer noticeable benefits.

### SUMMARY OF THE INVENTION

A hair care composition comprising a triglyceride oil having at least about 30% C18:1; and a low melting point wax.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

The term “molecular weight” or “M.Wt.” as used herein refers to the weight average molecular weight unless otherwise stated. The weight average molecular weight may be measured by gel permeation chromatography.

“QS” means sufficient quantity for 100%.

### Hair Products

As some consumers are interested in hair care compositions that do not contain silicones, there is a need for alternative conditioning actives. The hair care compositions of the present invention comprise a triglyceride natural oil to deliver conditioning performance. These oils are liquid at room temperature and are lubricious. However, to deliver conditioning from a rinse-out conditioner, the oils need to be delivered onto hair. There are two key parameters that control deposition of oils onto hair: i) interfacial properties of the oil vs. the hair surface; and ii) the viscosity of the oil.

The oil viscosity controls the kinetics of deposition and the rate of wetting. An increased viscosity will decrease the rate of spreading, but it also decreases “roll-up” rate, aiding durability during rinsing. Thus, a viscosity that is too low will allow the oil to be too easily removed, while a viscosity that is too high will mean the material will bounce off the surface and no spreading will occur. Thus, a problem to be solved is how to increase the viscosity of triglyceride oils to enable deposition on hair from a rinse-off product, but still retain conditioning lubricity and conditioning benefits.

The present inventors have discovered that a solution is to mix a triglyceride oil with a low melting point wax. This increases the viscosity but still allows for the conditioning benefits of the triglyceride. The low melting point waxes also provide some lubricity and conditioning. However, there is also a requirement that the oil/wax blends be homogeneous and stable, meaning that when the two are melted together, they do not separate back into the individual components upon cooling.

It has surprisingly been found that low melting point waxes and certain types of triglyceride oils can form a blend that is higher in viscosity than the original triglyceride oil while remaining a homogeneous blend upon cooling. This higher viscosity blend significantly increases deposition of triglyceride onto hair and delivers an enhanced conditioning performance as measured by wet and dry combing data. For example, Citrus aurantium dulcis (orange) peel wax and specific triglyceride oils form a higher viscosity blend that remains homogeneous. Not all triglyceride oils have the ability to increase viscosity when blended with Citrus aurantium dulcis (orange) peel wax. Important is the percentage of C18:1 (oleic acid) and C18:0 (stearic acid) in the oil blend. The present inventors have discovered that most effective is when the oleic acid proportion of the triglyceride is at least about 30%. In some embodiments, the oleic acid proportion of the triglyceride is at least about 50%, i.e., the triglyceride oil comprises at least about 50% C18:1. It is thought that the C18:1 chain of the triglyceride maximizes its positive hydrophobic-hydrophobic interaction with the ester side chains present in the Citrus aurantium dulcis (orange) peel wax, but also keeps the triglyceride fluid. A higher interaction would likely be present with a higher percentage of C18:0 side chains, but this also makes the triglyceride more solid and less lubricious.

### A. Cationic Surfactant System

The hair care composition described herein comprises a cationic surfactant system. The cationic surfactant system can be one cationic surfactant or a mixture of two or more cationic surfactants. The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amidoamine salt and mono-long alkyl quaternized ammonium salt.

The cationic surfactant system can be included in the hair care composition at a level of from about 0.1% to about 10%, alternatively from about 0.5% to about 8%, alternatively from about 0.8% to about 5%, and alternatively from about 1.0% to about 4%, by weight of the hair care composition.

### Mono-Long Alkyl Quaternized Ammonium Salt

The monoalkyl quaternized ammonium salt cationic surfactants useful herein are those having one long alkyl chain which has from 12 to 30 carbon atoms, from 16 to 24 carbon atoms, and in one embodiment at C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

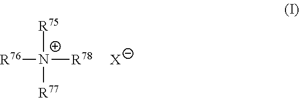

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, an/or 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, bchenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; in one embodiment l-glutamic acid, lactic acid, and/or citric acid. The amines herein can be partially neutralized with any of the acids at a molar ratio of the amine to the acid of from about 1:0.3 to about 1:2, and/or from about 1:0.4 to about 1:1.

### Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The dialkyl quaternized ammonium salt cationic surfactants useful herein are those having two long alkyl chains having 12-30 carbon atoms, and/or 16-24 carbon atoms, and/or 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

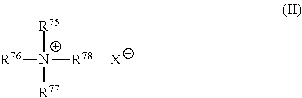

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ can be selected from an alkyl group of from 12 to 30 carbon atoms, from 16 to 24 carbon atoms, from 18 to 22 carbon atoms, and/or 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof. Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### B. High Melting Point Fatty Compound

The hair care composition comprises one or more high melting point fatty compounds. The one or more high melting point fatty compounds useful herein can have a melting point of 25° C. or higher, and can be selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are suitable for use in the hair care composition. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

High melting point fatty compounds of a single compound of high purity can be used. Single compounds of pure fatty alcohols selected from the group of pure cetyl alcohol, stearyl alcohol, and behenyl alcohol can also be used. By “pure” herein, what is meant is that the compound has a purity of at least about 90%, and/or at least about 95%. These single compounds of high purity provide good rinsability from the hair when the consumer rinses off the composition.

The one or more high melting point fatty compounds can be included in the hair care composition at a level of from about 0.1% to about 20%, alternatively from about 1% to about 15%, and alternatively from about 1.5% to about 8%, by weight of the hair care. The one or more high melting point fatty compounds can provide improved conditioning benefits such as slippery feel during the application of the hair care composition to wet hair, hair softness on dry hair, and moisturized feel on dry hair.

### C. Aqueous Carrier

The hair care composition comprises an aqueous carrier at a level of from about 75% to about 98%, alternatively from about 80% to about 95%, by weight of the hair care composition. Accordingly, the hair care composition can be in the form of pourable liquids (under ambient conditions). The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier can include water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

The hair care composition may have a pH in the range from about 2 to about 10, alternatively from about 3 to about 8, at 25° C. The hair care composition can also be effective toward washing out the existing minerals and redox metals deposits, which can reduce cuticle distortion and thereby reduce cuticle chipping and damage.

### D. Gel Matrix

The hair care composition can comprise a gel matrix. The gel matrix comprises a cationic surfactant, a high melting point fatty compound, and an aqueous carrier.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, from about 1:1 to about 1:10, and/or from about 1:1 to about 1:6.

### E. Additional Components

### 1. Natural Conditioning Agents

The hair care compositions herein may comprise a triglyceride oil blended with a wax. As discussed above, the triglyceride oil, while offering conditioning benefits, may not be effective by itself. There are certain viscosities that better enable deposition of the conditioner on the hair, such as 1.0 to 5.0 Pa at 1 1/s shear rate. Typical triglyceride oils have viscosity of 0.25-0.04 Pa at 1 1/s shear rate, which means that they can be easily removed during rinsing.

However, a triglyceride oil may be blended with a wax. The triglyceride oil mixed with a low melting point wax can have an increased viscosity but still retain conditioning benefits. The low melting point waxes can also provide some lubricity and conditioning. But it has been further discovered that some combinations of triglyceride oils and waxes work better than others. For example, specific triglyceride oils form a blend with Citrus aurantium dulcis (orange) peel wax that is homogeneous and higher in viscosity than the original triglyceride oil. This higher viscosity blend significantly increases deposition of the triglyceride onto hair and delivers enhanced conditioning performance. Important is the percentage of C18:1 (oleic acid) and C18:0 (stearic acid) in the oil blend. The oleic acid proportion of the triglyceride should be at least about 30% and in some embodiments at least about 50%. It is thought that the C18:1 chain of the triglyceride maximizes its positive hydrophobic-hydrophobic interaction with the ester side chains present in the Citrus aurantium dulcis (orange) peel wax, but still keeps the triglyceride fluid. A higher interaction would likely be present with a higher percentage of C18:0 side chains, but this also makes the triglyceride more solid and less lubricious.

In some embodiments, the triglyceride oil may be selected from the group consisting of safflower seed oil, avocado oil, almond oil, olive oil, tea seed oil, chullu (wild apricot) seed oil, peanut oil, marula oil and combinations thereof. These triglyceride oils are at least 30% C18:1, in some embodiments at least 50% C18:1. Safflower seed oil (high oleic acid (OA) version) has 73% C18:1; Avocado oil has from 55% to 75%, almond oil from 60% to 70%, and olive oil from 65% to 80% C18:1.

The ratio of triglyceride oil to the low melting point wax may be from about 90:10 to about 50:50, in some embodiments from about 85:15 to about 60:40. The amount of triglyceride in the hair care composition, by weight of the composition, may be from about 0.5% to about 5%, and in some embodiments from about 1% to about 3%.

While Citrus aurantium dulcis (orange) peel wax works well, other low melting point waxes may be used. The low melting point wax may be selected from the group consisting of Citrus aurantium dulcis (orange) peel wax, Citrus limon (lemon) peel wax, bayberry wax, lanolin wax, plant-derived lanolin wax, Aurantium amara (bitter orange) flower wax, Acacia farnesiana (cassie flower) wax, Narcissus poeticus (Narcissus flower) wax, Rosa centifolia (rose flower) wax, Jasminum sambac (jasmine) flower wax, and combinations thereof. Low melting in this case is considered to be a melting point of at most about 45° C.

In some embodiments, it may be an option to add a low level (less than about 10% by weight of the waxes) of a higher melting point wax to the low melting point wax to add additional viscosity. The final level of high melting point wax in the wax blend may be less than about 5%. Possible options for a high melting point wax include, but are not limited to, rice bran wax, sunflower wax, carnauba wax, candelilla wax, beeswax, hydrogenated triglyceride oils such as hydrogenated sunflower, soy bean, and olive oil, and any combinations thereof. In one embodiment, the wax blend may be sunflower wax and rice bran wax.

Table 1 below shows various triglyceride oils combined in a 75:25 ratio with Citrus aurantium dulcis (orange) peel wax (OPW), the triglyceride oil fatty acid content C18:1, and the blend's viscosity, measured at two different rates. This shows that the triglyceride oils with at least 30%, or at least 50%, C18:1 have higher viscosities when blended with the orange peel wax.

TABLE 1

| | | |
|---|---|---|
| Triglyceride | Fatty acid content | Viscosity (Pa) |

| 75:25 with OPW | C18:1 | 1 1/s | 10 1/s |
|---|---|---|---|
| High OA Safflower | 73% | 2.75 | 0.93 |
| Coconut | 5-8% | 1.22 | 0.54 |
| Jojoba | 9% | 0.54 | 0.30 |
| Avocado | 55-75% | 2.69 | 0.93 |
| Almond | 65-80% | 1.93 | 0.74 |

Table 2 below shows deposition data measured on virgin and colored hair for three hair conditioning compositions. The first hair care composition contained no wax and only safflower oil, at 3% by weight of the composition; the second hair care composition contained a 4:1 blend of safflower oil to orange peel wax, at 3% by weight of the composition; and the third hair care composition contained only the orange peel wax and no triglyceride, again at 3% by weight of the composition. The composition other than the 3% wax and/or oil was the same as Example 13 below. Using the test method described below, the amount of safflower oil deposited on the hair was measured. The data shows that the highest deposition resulted from the blend of safflower oil and orange peel wax. This was higher than when safflower oil was used alone. The hair care composition comprising only orange-peel wax naturally did not show any safflower oil deposition, yet the presence of the orange peel wax in the blend enabled higher safflower oil deposition. As expected, the deposition in general was greater for virgin hair compared to colored hair, but the deposition superiority of the blend vs. either the triglyceride or the wax alone is clear for both types of hair.

TABLE 2

| | | Safflower Deposition (μg/g) | |
|---|---|---|---|
| Sample Details | Hair Type | Std error in parenthesis | |
| 3% Safflower seed oil in | Virgin | 123.3 | (8.7) |
| conditioner | | | |
| 2.4% Safflower seed oil + | Virgin | 172.2 | (16.3) |
| 0.6% orange-peel wax in | | | |
| conditioner | | | |
| 3% Safflower seed oil in | Colored | 71.3 | (6.8) |
| conditioner | | | |
| 2.4% Safflower seed oil + | Colored | 96.89 | (11.4) |
| 0.6% orange-peel wax in | | | |
| conditioner | | | |

Tables 3 and 4 below show respectively the wet and dry combing data for the same three hair care compositions used in Table 2. For both wet and dry combing, the blend of safflower oil and orange peel wax results in the lowest friction. This demonstrates that the natural blend of safflower oil and orange peel wax can provide the consumer-desired benefits of a hair conditioner.

Tables 3 and 4:

| | | Wet Mid 1 | Wet Dry Mid 1 |
|---|---|---|---|
| | | Combing Force | Combing Force |
| | Hair | (gf) Std error | (gf) Std error |
| Sample Details | Type | in parenthesis | in parenthesis |
| 3% Safflower seed oil | Virgin | 84.75 (1.63) | 15.11 (0.32) |
| in conditioner | | | |
| 2.4% Safflower seed | Virgin | 71.83 (1.39) | 13.06 (0.22) |
| oil + 0.6% orange-peel | | | |
| wax in conditioner | | | |
| 3% Orange-peel wax | Virgin | 85.18 (1.11) | 16.07 (0.54) |
| in conditioner | | | |
| 3% Safflower seed oil | Colored | 83.20 (1.45) | 13.36 (0.26) |
| in conditioner | | | |
| 2.4% Safflower seed | Colored | 73.09 (1.67) | 12.39 (0.36) |
| oil + 0.6% orange-peel | | | |
| wax in conditioner | | | |
| 3% Orange-peel wax | Colored | 79.79 (1.12) | 13.21 (0.31) |
| in conditioner | | | |

In some embodiments, the hair care composition may be substantially free of or completely free of silicones. In still other embodiments, the blends of natural conditioning agents described herein may be combined with silicones or silicone conditioning agents.

The hair care composition can include a silicone conditioning agent which comprises a silicone compound. The silicone compound may comprise volatile silicone, non-volatile silicones, or combinations thereof. If volatile silicones are present, it will typically be incidental to their use as a solvent or carrier for commercially available forms of non-volatile silicone materials ingredients, such as silicone gums and resins. The silicone compounds may comprise a silicone fluid conditioning agent and may also comprise other ingredients, such as a silicone resin to improve silicone fluid deposition efficiency or enhance glossiness of the hair. The concentration of the silicone compound in the hair care composition typically ranges from about 0.01 wt % to about 10 wt %, from about 0.1 wt % to about 8 wt %, from about 0.1 wt % to about 5 wt %, or even from about 0.2 wt % to about 3 wt %. Suitable silicones for use herein include, but are not limited to PDMS (Dimethicone) silicones, PQAS (Silicone Quaternium-26); PDMS, terminal aminosilicone, and combinations thereof. In embodiments that comprise both natural conditioning agents and silicone conditioning agents, the ratio of natural conditioning agents to silicone conditioning agents may be from about 25:75 to about 75:25.

Additional suitable silicone compounds include (a) a first polysiloxane which is non-volatile, substantially free of amino groups, and has a viscosity of from about 100,000 mm²s⁻¹ to about 30,000,000 mm²s⁻¹; (b) a second polysiloxane which is non-volatile, substantially free of amino groups, and has a viscosity of from about 5 mm²s⁻¹ to about 10,000 mm²s⁻¹; (c) an aminosilicone having less than about 0.5 wt % nitrogen by weight of the aminosilicone; (d) a silicone copolymer emulsion with an internal phase viscosity of greater than about 100×10⁶ mm²s⁻¹, as measured at 25° C.; (c) a silicone polymer containing quaternary groups; or (f) a grafted silicone polyol, wherein the silicone compounds (a)-(f) are disclosed in U.S. Patent Application Publication Nos. 2008/0292574, 2007/0041929, 2008/0292575, and 2007/0286837, each of which is incorporated by reference herein in its entirety. Further descriptions of suitable silicone conditioning agents may be found in U.S. Application No. 62/945,959, Attorney Docket No. 15687P.

### 2. Other Conditioning Agents

Also suitable for use in the hair care compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586, 4,507,280, 4,663,158, 4,197,865, 4,217,914, 4,381,919, and 4,422,853.

a. Organic Conditioning Oils

The hair care composition may also further comprise an organic conditioning oil. According to embodiments, the hair care composition may comprise from about 0.05 wt % to about 3 wt %, from about 0.08 wt % to about 1.5 wt %, or even from about 0.1 wt % to about 1 wt %, of at least one organic conditioning oil as the conditioning agent, in combination with other conditioning agents described herein. Suitable conditioning oils include hydrocarbon oils, polyolefins, and fatty esters. Suitable hydrocarbon oils include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils are typically from about C12 to about C19. Branched chain hydrocarbon oils, including hydrocarbon polymers, typically will contain more than 19 carbon atoms. Suitable polyolefins include liquid polyolefins, liquid poly-α-olefins, or even hydrogenated liquid poly-α-olefins. Polyolefins for use herein may be prepared by polymerization of C4 to about C14 or even C6 to about C12. Suitable fatty esters include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

### 3. Nonionic Polymers

The hair care composition may also further comprise a nonionic polymer. According to an embodiment, the conditioning agent for use in the hair care composition of the present invention may include a polyalkylene glycol polymer. For example, polyalkylene glycols having a molecular weight of more than about 1000 are useful herein. Useful are those having the following general formula (VIII):

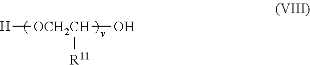

wherein R¹¹ is selected from the group consisting of H, methyl, and mixtures thereof; and v is the number of ethoxy units. The polyalkylene glycols, such as polyethylene glycols, can be included in the hair care compositions of the present invention at a level of from about 0.001 wt % to about 10 wt %. In an embodiment, the polyethylene glycol is present in an amount up to about 5 wt % based on the weight of the composition. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

### 4. Suspending Agent

The hair care composition may further comprise a suspending agent at concentrations effective for suspending water-insoluble material in dispersed form in the compositions or for modifying the viscosity of the composition. Such concentrations range from about 0.1 wt % to about 10 wt %, or even from about 0.3 wt % to about 5.0 wt %.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carrageenan, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethylencimine, and inorganic water soluble material such as bentonite, aluminum magnesium silicate, laponite, hectonite, and anhydrous silicic acid.

Commercially available viscosity modifiers highly useful herein include Carbomers with trade names Carbopol® 934, Carbopol® 940, Carbopol® 950, Carbopol® 980, and Carbopol® 981, all available from B. F. Goodrich Company, acrylates/steareth-20 methacrylate copolymer with trade name ACRYSOL™ 22 available from Rohm and Hass, nonoxynyl hydroxyethylcellulose with trade name Amercell™ POLYMER HM-1500 available from Amerchol, methylcellulose with trade name BENECEL®, hydroxyethyl cellulose with trade name NATROSOL®, hydroxypropyl cellulose with trade name KLUCEL®, cetyl hydroxyethyl cellulose with trade name POLYSURF® 67, all supplied by Hercules, ethylene oxide and/or propylene oxide based polymers with trade names CARBOWAX® PEGs, POLYOX WASRs, and UCON® FLUIDS, all supplied by Amerchol.

Other optional suspending agents include crystalline suspending agents which can be categorized as acyl derivatives, long chain amine oxides, and mixtures thereof. These suspending agents are described in U.S. Pat. No. 4,741,855.

These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

### H. Benefit Agents

The hair care composition can further comprises one or more additional benefit agents. The benefit agents comprise a material selected from the group consisting of anti-dandruff agents, vitamins, lipid soluble vitamins, chelants, perfumes, brighteners, enzymes, sensates, attractants, anti-bacterial agents, dyes, pigments, bleaches, and mixtures thereof.

In one aspect said benefit agent may comprise an anti-dandruff agent. Such anti-dandruff particulate should be physically and chemically compatible with the components of the composition, and should not otherwise unduly impair product stability, aesthetics or performance.

According to an embodiment, the hair care composition comprises an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt.

Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt % to about 5 wt %, or from about 0.1 wt % to about 3 wt %, or from about 0.1 wt % to about 2 wt %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. Nos. 2,809,971; 3,236,733; 3,753,196; 3,761,418; 4,345,080; 4,323,683; 4,379,753; and 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, fcharcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline clioquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active can be included in an amount of from about 0.01 wt % to about 5 wt %, or from about 0.1 wt % to about 3 wt %, or from about 0.3 wt % to about 2 wt %. In an embodiment, the azole anti-microbial active is ketoconazole. In an embodiment, the sole anti-microbial active is ketoconazole.

Embodiments of the hair care composition may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the hair care composition comprises an effective amount of a zinc-containing layered material. In an embodiment, the hair care composition comprises from about 0.001 wt % to about 10 wt %, or from about 0.01 wt % to about 7 wt %, or from about 0.1 wt % to about 5 wt % of a zinc-containing layered material, by total weight of the hair care composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+} A^{m-}_{x/m}·nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺ Aⁿ⁻_{(1=3y)/n}·nH₂O where the two metal ions (M²⁺) may be the same or different. If they are the same and represented by zinc, the formula simplifies to [Zn₁₊ₓ(OH)₂]^{2x+} 2x A⁻·nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replaces the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

### Product Forms

The compositions of the present invention can be in the form of rinse-off products or leave-on products, and can be formulated in a wide variety of product forms, including but not limited to creams, gels, emulsions, mousses and sprays. The composition of the present invention is especially suitable for conditioning compositions especially leave-on, leave-in, and/or no-rinse compositions. Leave-on and leave-in conditioning compositions are generally used on dry, semi-wet, and/or wet hair without rinsing out the composition. By no-rinse compositions, what is meant herein is a hair care composition used on semi-wet to wet hair after shampooing, without rinsing out the no-rise hair care composition (such as a no-rinse hair conditioner).

### Test Methods

### Rheology Method

A TA instruments rheometer was used (Discovery HR-3) to perform a shear rate vs viscosity sweep. A 40 mm parallel steel plate was used at a temperature of 25° C. The shear rate sweep was from 0.01 to 1000 1/s with 10 points per decade and a sample period of 10 secs. The data report was at 1 1/s and 10 1/s which are relevant shear rates for product application and rinsing.

### Deposition Method

For each sample, approximately 0.5 g of hair was cut in greater than 40 mm segments into vials. First the hair was extracted gently with hexane. The hexane extraction consists of extracting the hair with hexane two times then concentrating the dried residue in 2:1 chloroform:methanol and analyzed by gas chromatography (GC). Next a more aggressive extraction using 2:1 then 1:1 chloroform:methanol was used. The chloroform contained 10 mM dimethylhexylamine (DMHA) and the methanol 1% formic acid. Each extraction was heated for 30 mins at 65° C. with the hair and then combined and the dried residue re-dissolved in a 2:1 chloroform:methanol then analyzed by GC.

Safflower seed oil was quantified by GC with flame ionization detection using a polydimethylsiloxane capillary column with hydrogen mobile phase. Trinonadecanoin was used as an internal standard with a calibration curve.

### Hair Treatment and Combing Method

Tresses of hair 8 inch in length and 4 g in weight were used for deposition and combing testing; three tresses per treatment leg. A non-conditioning shampoo was applied at a dose of 0.1 g/g hair and lathered for 30 secs followed by a 30 sec rinse. Excess water was squeezed from the tresses and then conditioner with or without the oil blend was applied at a dose of 0.1 g/g hair and milked for 30 secs followed by a 30 sec rinse. The hair was then dried in a hot box at ˜65° C. for 30 minutes. This protocol was repeated for a total of six cycles.

The tresses are tested for force it takes to comb through each tress on an Instron (Model 5564) with a 50N lead cell. The tresses are pre-combed before the measurement and then positioned in a set-up with 2 parallel combs (fine tines) that are 7.2 cm apart. The hair is then pulled through the two combs and the force exerted on the load cell measured. This is repeated five times. This measurement is performed wet and dry with hair being left for 24 hours in a controlled humidity room at 50% RH and 22° C. The force Mid 1 is friction of both combs through bulk of hair, Mid 2 is friction of the top comb through the bulk of hair.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Smoothing Rinse-Off Conditioner Compositions

| | Ex. 1 | Ex. 2 | Ex. 3 | Ex. 4 |
|---|---|---|---|---|
| Components | Wt % | Wt % | Wt % | Wt % |
| Stearyl alcohol | 2.32 | 2.32 | 2.32 | 2.32 |
| Cetyl alcohol | 0.93 | 0.93 | 0.93 | 0.93 |
| Dicetyldimonium chloride | 0.34 | 0.34 | 0.34 | 0.34 |
| Behentrimonium methosulfate | 1.16 | 1.16 | 1.16 | 1.16 |
| Propylene glycol | 0.16 | 0.16 | 0.16 | 0.16 |
| Isopropyl alcohol | 0.28 | 0.28 | 0.28 | 0.28 |
| Disodium EDTA Dihydrate | 0.13 | 0.13 | 0.13 | 0.13 |
| Terminal amodimethicone¹ | 0.75 | 0.75 | 0.75 | 0.75 |
| Safflower Seed Oil | 2.4 | 0.85 | — | 1.5 |
| Avocado Oil | — | — | 0.80 | — |
| Citrus Aurantium Dulcis (Orange) | 0.6 | 0.15 | 0.20 | 0.5 |
| Peel Wax | | | | |
| Methylchloroisothiazolinone/ | 0.0005 | 0.0005 | 0.0005 | 0.0005 |
| Methylisothiazolinone² | | | | |
| Benzyl alcohol | 0.40 | 0.40 | 0.40 | 0.40 |
| Distilled Water | Q.S. | Q.S. | Q.S. | Q.S. |

¹Terminal amodimethicone with visc. Of 10,000 cP at 25° C. is available by Momentive Performance Materials.
²Kathon CG available from Dow (1.5 wt % active)

### Volumizing Rinse-Off Conditioner Compositions

| | Ex. 5 | Ex. 6 | Ex. 7 | Ex. 8 |
|---|---|---|---|---|
| Components | Wt % | Wt % | Wt % | Wt % |
| Hydroxypropyl guar¹ | 0.35 | 0.35 | 0.35 | 0.35 |
| DTDMAC (Quaternium-18²) | 0.75 | 0.75 | 0.75 | 0.75 |
| Stearamidopropyldimethylamine | 1.00 | 1.00 | 1.00 | 1.00 |
| Glyceryl monostearate | 0.25 | 0.25 | 0.25 | 0.25 |
| Emulsifying wax NF (Polywax | 0.50 | 0.50 | 0.50 | 0.50 |
| NF) | | | | |
| Cetyl alcohol | 1.20 | 1.20 | 1.20 | 1.20 |
| Stearyl alcohol | 0.80 | 0.80 | 0.80 | 0.80 |
| Oleyl alcohol | 0.25 | 0.25 | 0.25 | 0.25 |
| Citric acid | 0.13 | 0.13 | 0.13 | 0.13 |
| EDTA | 0.10 | 0.10 | 0.10 | 0.10 |
| Terminal amodimethicone³ | 0.50 | 0.50 | 0.50 | 0.50 |
| Safflower Seed Oil | 2.4 | 0.85 | — | 1.5 |
| Almond Oil | — | — | 0.80 | — |
| Citrus Aurantium Dulcis (Orange) | 0.6 | 0.15 | 0.20 | 0.5 |
| Peel Wax | | | | |
| Methylchloroisothiazolinone/ | 0.0005 | 0.0005 | 0.0005 | 0.0005 |
| Methylisothiazolinone⁴ | | | | |
| Benzyl alcohol | 0.4 | 0.4 | 0.4 | 0.4 |
| Distilled Water | Q.S. | Q.S. | Q.S. | Q.S. |

¹Jaguar HP-105 supplied by Rhodia
²Diatallowdimethylammonium chloride
³Terminal amodimethicone with visc. Of 10,000 cP at 25° C. is available by Momentive Performance Materials.
⁴Kathon CG available from Dow (1.5 wt % active).

### Hair Repair Rinse-Off Conditioner Compositions

| | Ex. 9 | Ex. 10 | Ex. 11 | Ex. 12 |
|---|---|---|---|---|
| Components | Wt % | Wt % | Wt % | Wt % |
| Behentrimonium chloride | 2.28 | 2.28 | 2.28 | 2.28 |
| Stearyl alcohol | 4.64 | 4.64 | 4.64 | 4.64 |
| Cetyl alcohol | 0.93 | 0.93 | 0.93 | 0.93 |
| Isopropyl alcohol | 0.57 | 0.57 | 0.57 | 0.57 |
| Disodium EDTA Dihydrate | 0.13 | 0.13 | 0.13 | 0.13 |
| Dimethicone¹ | 4.20 | 4.20 | 4.20 | 4.20 |
| Sodium hydroxide | 0.02 | 0.02 | 0.02 | 0.02 |
| Safflower Seed Oil | 2.4 | 0.85 | — | 1.5 |
| Olive Oil | — | — | 0.80 | — |
| Citrus Aurantium Dulcis (Orange) | 0.6 | 0.15 | 0.20 | 0.5 |
| Peel Wax | | | | |
| Methylchloroisothiazolinone/ | 0.0005 | 0.0005 | 0.0005 | 0.0005 |
| Methylisothiazolinone² | | | | |
| Benzyl alcohol | 0.40 | 0.40 | 0.40 | 0.40 |
| Distilled Water | Q.S. | Q.S. | Q.S. | Q.S. |

¹Mixture of silicone gum and silicone oil XF49-B1747 available from Momentive Performance Materials.
²Kathon CG available from Dow (1.5 wt % active).

### Moisture Rinse-Off Conditioner

| | Ex. 13 | Ex. 14 | Ex. 15 | Ex. 16 |
|---|---|---|---|---|
| Components | Wt % | Wt % | Wt % | Wt % |
| sodium Benzoate | 0.200 | 0.200 | 0.200 | 0.200 |
| L-glutamic acid | 1.55 | 1.55 | 1.55 | 1.55 |
| Citric acid | 0.25 | 0.25 | 0.25 | 0.25 |
| Behenamidopropyl Dimethylamine | 3.74 | 3.74 | 3.74 | 3.74 |
| (BAPDMA) | | | | |
| Cetyl Alcohol (95%) | 3.83 | 3.83 | 3.83 | 3.83 |
| Steary Alcohol | 2.85 | 2.85 | 2.85 | 2.85 |
| Shea Butter | 0.50 | 0.50 | 0.50 | 0.50 |
| Safflower Seed Oil | 2.4 | 0.85 | — | 1.5 |
| Peanut Oil | — | — | 0.80 | — |
| Citrus Aurantium Dulcis (Orange) | 0.6 | 0.15 | 0.20 | 0.5 |
| Peel Wax | | | | |
| Distilled water | Q.S. | Q.S. | Q.S. | Q.S. |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
a gel matrix comprising a cationic surfactant, a high melting point fatty compound, and an aqueous carrier;
a triglyceride oil having at least about 30 wt. % C18:1; and
about 5% or less, by weight of the composition, of a low melting point wax having a melting point of about 45° C. or less, wherein the ratio of triglyceride oil to low melting point wax is about 90:10 to about 50:50.

2. The hair care composition of claim 1, wherein the ratio of triglyceride oil to low melting point wax is about 85:15 to about 60:40.

3. The hair care composition of claim 1, wherein the composition comprises from about 0.5% to about 5%, by weight of the composition, of triglyceride oil.

4. The hair care composition of claim 1, wherein the composition comprises from about 1% to about 3%, by weight of the composition, of triglyceride oil.

5. The hair care composition of claim 1, wherein the triglyceride oil is selected from the group consisting of safflower seed oil, avocado oil, almond oil, olive oil, tea seed oil, chullu (wild apricot) seed oil, peanut oil, marula oil and combinations thereof.

6. The hair care composition of claim 1, wherein the low melting point wax is selected from the group consisting of Citrus aurantium dulcis (orange) peel wax, Citrus limon (lemon) peel wax, bayberry wax, lanolin wax, plant-derived lanolin wax, Aurantium amara (bitter orange) flower wax, Acacia farnesiana (cassie flower) wax, Narcissus poeticus (Narcissus flower) wax, Rosa centifolia (rose flower) wax, Jasminum sambac (jasmine) flower wax, and combinations thereof.

7. The hair care composition of claim 1, wherein the low melting point wax is selected from the group consisting of Citrus aurantium dulcis (orange) peel wax, Citrus limon (lemon) peel wax, Aurantium amara (bitter orange) flower wax, bayberry wax, lanolin wax, and combinations thereof.

8. The hair care composition of claim 1, wherein the fatty alcohol is selected from the group consisting of cetyl alcohol, stearyl alcohol, behenyl alcohol, and mixtures thereof.

9. The hair care composition of claim 8, wherein the fatty alcohol is present at a level of from about 0.1 wt % to about 20 wt %.

10. The hair care composition of claim 1, wherein the hair care composition is a hair conditioner.

11. The hair care composition of claim 10, wherein the hair care composition is a leave on treatment.

13. The hair care composition of claim 10, wherein the hair care composition is a rinse off conditioner.

14. The hair care composition of claim 1, wherein the cationic surfactant is selected from the group consisting of mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amidoamine salt, mono-long alkyl quaternized ammonium salt and combinations thereof.

15. The hair care composition of claim 1, wherein the composition comprises from about 0.1 wt % to about 10 wt % of the cationic surfactant.

16. The hair care composition of claim 1, wherein the triglyceride oil has at least about 50% C18:1.
